(19) United States
(12) Patent Application Publication
(10) Pub. No.: US 2007/0237716 A1
(43) Pub. Date: Oct. 11, 2007

(54) COMPOSITIONS AND METHODS FOR REVERSE TRANSCRIPTASE-POLYMERASE CHAIN REACTION (RT-PCR) OF HUMAN B-RETROVIRUS

(76) Inventors: Andrew L. Mason, Edmonton (CA); Lizhe Xu, Groton, CT (US)

Correspondence Address: WINSTON & STRAWN LLP;PATENT DEPARTMENT, 1700 K STREET, N.W., WASHINGTON, DC, 20006, US

(21) Appl. No.: 11/565,289

(22) Filed: Nov. 30, 2006

Related U.S. Application Data
(60) Provisional application No. 60/741,290, filed on Nov. 30, 2005.

(51) Int. Cl. A61K 49/00; C12Q 1/68

(52) U.S. Cl. 424/9.1; 435/6

(57) ABSTRACT

The invention can be summarized as follows. The present invention provides compositions, methods and kits useful for the amplification of nucleic acid molecules from human β-retrovirus by reverse transcriptase-polymerase chain reaction (RT-PCR). Specifically, the invention provides compositions and methods for the amplification of nucleic acid molecules in a one or two-step real time RT-PCR procedure using reverse transcriptase, DNA polymerase, or a combination of both enzymes. The invention provides for the rapid and efficient amplification, detection and quantification of human β-retrovirus nucleic acids.

This application claims the benefit of provisional application No. 60/741,290 filed Nov. 30, 2005.

### TECHNICAL FIELD

The present invention relates to compositions, methods and kits for performing reverse transcriptase polymerase chain reaction (RT-PCR).

### BACKGROUND OF THE INVENTION

### PCR Amplification of RNA

Reverse transcriptases and methods for using them are known in the art for reverse transcribing RNA prior to PCR amplification. These methods, often collectively referred to as reverse transcriptase-PCR (RT-PCR), are widely used for detection and quantification of RNA.

While many RT-PCR, methods are known in the art, the efficient amplification of a target nucleic acid cannot be accomplished by a single standard protocol. In most cases, a variety of technical considerations and other problems are present that result in less than optimal amplification of target nucleic acids. In an attempt to address some of the technical problems often associated with RT-PCR, a number of protocols have been developed taking into account the basic steps of the procedure: (a) the isolation of RNA and the hybridization of reverse primer to the target nucleic acid; (b) the synthesis of cDNA; and (c) PCR amplification.

In a two-step RT-PCR. procedure, reverse transcription is performed as an independent step, preferably using the optimal buffer condition for reverse transcriptase activity. Typically after cDNA synthesis, the reaction mixture is diluted to decrease the concentration of salts, for example MgCl₂ and other components, such as deoxyribonucleoside triphosphate (dNTP) concentrations. The resulting conditions are better suited for Taq DNA Polymerase activity, and PCR is carried out according to standard conditions (see for example U.S. Pat. No. 4,683,195 and U.S. Pat. No. 4,683,202 which are hereby incorporated by reference). In contrast to the two step procedure, one step RT-PCR methods usually employ a common buffer for both reverse transcriptase and Taq DNA Polymerase activities.

Attempts to enhance or streamline the process of RT-PCR have been difficult, and often the end results depend on factors such as, but not limited to the choice of primers and target nucleic acid to be amplified, the reaction and amplification conditions, the biological sample and relative abundance of the target nucleic acid in the biological sample. For example, the efficient detection, amplification and quantification of viral sequences from biological tissues and fluids can be difficult.

Primary Biliary Cirrhosis and Autoimmunity

Primary biliary cirrhosis (PBC) is a progressive pluriglandular disease affecting the liver, pancreas, salivary and lachrymal glands (Neuberger, 1997, Lancet 850:875-79; Epstein et al., 1980, Lancet 1:1166-68). The hepatic disease is characterized by a florid bile duct lesion with lymphocytic infiltration and granulomatous destruction of 30 to 80 μm sized interlobular bile ducts (Rubin et al., 1965, Am. J. Pathol. 46:387-407). There is no curative therapy, apart from liver transplantation, and patients usually develop cirrhosis (Neuberger et al., 1997, Lancet 250:875-879). It is estimated to account for approximately 2% of patients dying from cirrhosis in Europe and 10% of patients that requiring orthotopic liver transplantation in North America (Neuberger et al., 1997, Lancet 250:875-879).

The disease has been observed in all races and predominantly affects women (Neuberger, 1997, Lancet 350: 875-879). To date, non-HLA genetic factors predisposing to PBC have not been identified but a positive family history provides the greatest risk of developing disease (Sherlock et al., 1993, Primary biliary cirrhosis: definition and epidemiological features. Kluwer Academic Publishers, Doredrecht/Boston/London, pp. 341-49). There are well documented cases of clustering in families and one report documented a 2.4% familial prevalence (Sherlock et al., 1993, Primary biliary cirrhosis: definition and epidemiological features. Kluwer Academic Publishers, Doredrecht/Boston/London. 341-49 pp). No HLA class I alleles are associated with PBC but other immunogenetic factors appear to play an important role.

We have explored an infectious etiology of PBC. In the first instance, we used representational difference analysis to identify retroviral sequences in the liver of a patient with PBC, we found that the majority of patients with PBC had antibody reactivity to a retrovirus isolated from patients with Sjögren's syndrome and then cloned a retrovirus pol gene sequence from a PBC biliary epithelial cells cDNA library (Mason, A et al., Lancet 1998; 351:1620-24; Xu et al., Proc. Natl. Acad. Sci 2003; 100:8454-8459.). By BLASTN search, individual clones had a variable 91%-97% nucleotide homology with the mouse mammary tumor virus (MMTV) and with retroviral sequences derived from human breast cancer samples (Xu L, et al., Hepatology 2004; 39:151-156). The human β-retrovirus is flanked by 2 long terminal repeat regions and contains 5 potential colinear open reading frames of 100 or more codons that encode Gag, protease (Pro), polymerase (Pol), envelope (Env) and superantigen (Sag) proteins. Like MMTV, a −1 frame shift is required to generate the Gag-Pro polyprotein and a second −1 frame shift is required to generate the Gag-Pro-Pol polyprotein, whereas the env and sag genes are translated as individual open reading frames (Xu L, et al., Hepatology 2004; 39:151-156).

In reviewing the results from pilot studies of single and combination antiretroviral therapy in patients with primary biliary cirrhosis (Mason et al., Am. J. Gastroenterol 2004; 99:2348-2355 which is hereby incorporated by reference), the authors subject serum samples from patients to RT-PCR before and after therapy. Using this technique, it was shown that 5 of 9 patients taking lamivudine had detectable virus in the serum prior to treatment and 4 had detectable virus in serum after treatment. While such a method may be used to determine if infection is eradicated or present in a subject, it does not suggest whether the present course of treatment may be working. Indeed, the authors note that the lack of sensitive and qualitative detection assays to link diminishing viral load with significant clinical improvements is a shortfall.

There is a need in the art for optimized assays for detecting, amplifying, diagnosing and quantifying human β-retrovirus nucleic acids in biological samples. Further, there is a need in the art for optimized RT-PCR assays for detecting, amplifying, diagnosing and quantifying human retrovirus nucleic acids in human biological samples.

It is an object of the invention to overcome disadvantages of the prior art.

The above object is met by the combinations of features of the main claims, the sub-claims disclose further advantageous embodiments of the invention.

### SUMMARY OF THE INVENTION

The present invention relates to compositions, methods and kits for performing reverse transcriptase polymerase chain reaction (RT-PCR).

According to an embodiment of the present invention, there is provided a method of monitoring the therapy of a subject having a β-retrovirus infection comprising,
a) treating the subject with one or more therapeutics, and;
b) quantifying the level of human β-retrovirus in said subject.

In a preferred embodiment, the quantifying is performed by real time RT-PCR.

The present invention also contemplates a method as defined above, wherein the one or more therapeutics comprise lamivudine, zidovudine, combivir, ursodeoxycholic acid or a combination thereof.

The present invention also contemplates a method as defined above, wherein the real time RT-PCR employs a primer and probe as defined in Table 1. In a further embodiment, the RT-PCR is performed on nucleic acids obtained from a biological sample, such as for example a tissue, fluid or combination thereof isolated from a human subject. In a preferred embodiment, the biological sample is blood. In an alternate embodiment, which is not meant to be limiting, the biological sample is liver tissue from a biopsy.

The present invention also provides a method of monitoring the therapy of a subject having or suspected of having a human β-retrovirus infection comprising:
a) quantifying one or more pretreatment levels of human β-retrovirus in the subject;
b) treating the subject with one or more therapeutics, and;
c) quantifying one or more post-treatment levels of human β-retrovirus in the subject.
   Further, it is contemplated that during said b) treating said subject with one or more therapeutics, one or more levels of human β-retrovirus also may be determined.

The present invention also provides a method as defined above wherein the level of human β-retrovirus is monitored in combination with one or more enzyme or blood/tissue component levels, for example, but not limited to bilirubin, serum amylase, serum bicarbonate, lactate, alkaline phosphatase, aspartate aminotransferase (AST), alanine aminotransferase (AIFT) or a combination thereof.

The present invention also provides a method of screening a compound or composition for anti-human β-retrovirus activity comprising,
a) administering the compound or composition to one or more subjects having human β-retrovirus;
b) quantifying the level of human β-retrovirus in said subject.

In the method as defined above, human β-retrovirus infection may detected and/or quantified prior to said a) administering the compound or composition to one or more subjects.

The present invention also provides a kit for detecting mid/or quantifying human β-retrovirus in a biological sample comprising,
a) one or more primers for amplifying a human β-retrovirus target sequence,
b) one or more reverse transcriptases,
c) one or more DNA polymerases,
d) one or more probes for performing real time RT-PCR,
e) one or more buffers for performing, terminating, inhibiting, diluting, washing one or more enzymatic reactions, or products produced or purified in a RT-PCR reaction;
f) one or more restriction enzymes for cleaving DNA amplified from a PCR reaction,
g) one or more hybridization probes for identifying DNA amplified from a PCR reaction,
h) instructions for performing a RT-PCR reaction using the components of the kit,
i) one or more therapeutics;
j) one or more dNTPs, or the like,
   or any combination thereof.

In a preferred embodiment, which is not meant to be limiting , the kit comprises primers that hybridize to the U5 long terminal repeat or pol regions of the human β-retrovirus. The primers may be as defined in Table 1.

This summary of the invention does not necessarily describe all necessary features of the invention but that the invention may also reside in a sub-combination of the described features.

### BRIEF DESCRIPTION OF THE DRAWINGS

These and other features of the invention will become more apparent from the following description in which reference is made to the appended drawings wherein:

FIG. 1 shows a standard curve of real time RT-PCR assay for the U5 primers demonstrating a linear range from about 4×10⁵ to about 4 DNA copies.

FIG. 2 shows a standard curve of real time RT-PCR assay for the pol primers.

FIG. 3 shows the detection of human β-retrovirus RNA in samples derived from patients with PBC versus those from control non-PBC subjects. In panel (a), Ct values from 6 PBC patients' serum samples varied from 34.83 and 35.86 to Ct<=37, or negative. In panel (b) Ct values from liver sample of PBC patients (n=2, Ct=28.49 and 30.06) and non-PBC subjects (n=6, Ct<=37, or negative) and not in control samples.

FIG. 4 shows a graph of human :-retrovirus levels assessed by real-time RT-PCR in a patient with PBC unresponsive to UDCA treatment for 4 years (estimated viral copy number in Meq/ml shown by lower graph line). The subject experienced a partial biochemical response in viral load, ALT and alkaline phosphatase levels after 3 months of lamivudine therapy but then relapsed. Complete loss of virus with normalization of liver biochemistry was seen with Combivir treatment but biochemical and virological rebound was seen at month 9 of Combivir therapy.

### DESCRIPTION OF PREFERRED EMBODIMENT

The present invention relates to compositions and methods for performing reverse transcriptase polymerase chain reaction (RT-PCR).

The following description is of a preferred embodiment by way of example only and without limitation to the combination of features necessary for carrying the invention into effect.

The present invention is directed to compositions, methods and kits for detecting human β-retrovirus including, but not limited to amplification of nucleic acid molecules from human β-retrovirus by reverse transcriptase-polymerase chain reaction (RT-PCR), and identification of human β-retrovirus proteins and antibodies thereto by enzyme linked immunosorbant assays (ELISAs) or the like.

In an embodiment, the present invention is directed to compositions, methods and kits for use in reverse transcriptase-polymerase chain reaction (RT-PCR) for detection, production, amplification, analysis and quantification of human β-retrovirus nucleic acids from biological samples. In particular, the invention provides compositions comprising a variety of components in various combinations. Such components may include, but are not limited to, one or more enzymes having reverse transcriptase activity, one or more DNA polymerases, one or more primers, one or more probes, one or more nucleotides, one or more buffers, or a combination thereof. These compositions may be used in the methods and kits of the invention to identify, diagnose, produce, amplify, analyze, quantitate and otherwise manipulate human β-retrovirus nucleic acid molecules using a one- or two-step RT-PCR procedure. In addition, the composition, methods and kits as provided by the present invention may be employed for genetic testing and/or the prognosic evaluation of human β-retrovirus infection prior to therapy, during therapy, post therapy, or any combination thereof.

As used herein, the term “human β-retrovirus nucleic acids” refers to RNA from the human beta retrovirus viral genome, for example, as described in Xu et al., Hepatology 2004; 39:151-156, fragments thereof, transcripts thereof, and mutant sequences derived therefrom. Additional information concerning human β-retrovirus nucleic acids and sequences may be found in Example 1.

As used herein, a “biological sample” refers to a sample of tissue or fluid isolated from an individual. Thus, “biological sample” includes but is not limited to, plasma, serum, spinal fluid, lymph fluid, the external sections of the skin, respiratory, intestinal, and genitourinary tracts, tears, saliva, milk, blood cells, tumors, organs, and also samples of in vitro cell culture constituents (including, but not limited to conditioned medium resulting from the growth of cells in cell culture medium, putatively virally infected cells, recombinant cells, and cell components).

Buffers

The one or more buffers in the compositions of the invention provide appropriate pH and ionic conditions for the enzymes as described herein. In an embodiment, which is not meant to be limiting, the enzymes may include one or more reverse transcriptases, one or more DNA polymerases, or a combination thereof. It is also contemplated that nucleotides may used in the compositions, for example, but not limited to deoxyribonucleoside triphosphates (dNTPs), and the primer nucleic acid molecules capable of hybridizing to the human β-retrovirus nucleic acids molecules act as substrates for the synthesis or amplification of nucleic acid molecules in accordance with an embodiment of the invention. The compositions of the invention may also include additional components as described herein.

Reverse Transcriptase Enzymes

The compositions of the present invention may also comprise enzymes having reverse transcriptase activity. Any reverse transcriptase enzyme may be employed provided that it is capable of reverse transcribing human β-retrovirus RNA following binding of the primer to the human β-retrovirus primer target sequence. Enzymes having reverse transcriptase activity are commercially available. For example, but not wishing to be limiting in any manner, SUPERSCRIPT™, SUPERSCRIPT II™, Moloney Murine Leukemia Virus reverse transcriptases (M-MLV), and RSV reverse transcriptase are available from Life Technologies, Inc. (Rockville, Md.). Any one or combination of reverse transcriptases enzymes may be contained or employed in the compositions, methods and kits of the present invention. In an embodiment of the present invention, which is not meant to be limiting, the enzymes exhibit reduced RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)).

By an enzyme “reduced in RNase H activity” it is meant that the enzyme has less than about 20%, more preferably less than about 15%, 10% or 5%, and most preferably less than about 2%, of the RNase H activity of a wildtype enzyme under specific conditions. The RNase H activity may be determined by a variety of assays, such as those described, for example, in U.S. Pat. No. 5,244,797, in Kotewicz, M. L., et al., Nucl. Acids Res. 16:265 (1988) and in Gerard, G. F., et al., FOCUS 14(5):91 (1992), the disclosures of all of which are fully incorporated herein by reference.

DNA Polymerases

The compositions of the invention may also comprise one or more DNA polymerases, which are preferably thermostable DNA polymerases. These DNA polymerases may be isolated from natural or recombinant sources, by techniques that are well-known in the art (See for example, but not limited to WO 92/06200, U.S. Pat. No. 5,455,170, U.S. Pat. No. 5,466,591, WO 96/10640), from a variety of thermophilic bacteria that are available commercially (for example, but not limited to American Type Culture Collection (Rockville, Md.) or may be obtained by recombinant DNA techniques as described for example, but not limited to in WO 96/10640). Without wishing to be limiting, suitable sources of thermostable polymerases or the genes thereof for expression in recombinant systems are the thermophilic bacteria Thermus thermophilus, Thermococcus litoralis, Pyrococcus furiosus, Pyrococcus woosii and other species of the Pyrococcus genus Bacillus sterothermophilus, Sulfolobus acidocaldarius, Thermoplasma acidophilum, Thermus flavus, Thermus ruber, thermus brockianus, Thermotoga neapolitana, Thermotoga maritima and other species of the Thermotoga genus, and Methanobacterium thermoautotrophicum, and mutants, variants or derivatives thereof. It is to be understood, however, that thermostable DNA polymerases from other organisms may also be used in the present invention without departing from the scope or preferred embodiments thereof. As an alternative to isolation, thermostable DNA polymerases are available commercially from, for example, but not limited to Life Technologies, Inc. (Rockville, Md.), New England BioLabs (Beverly, Mass.), Finnzymes Oy (Espoo, Finland), Stratagene (La Jolla, Calif.), Boehringer Mannheim Biochemicals (Indianapolis, Ind.) and Perkin Elmer Cetus (Norwalk, Conn.).

In a preferred embodiment, the thermostable DNA polymerase or polymerases for use in the compositions, methods and kits of the present invention include, but are not limited to, Taq, Tne, Tma, Tli/VENT™, DEEPVENT™, Pfu, Pwo, Tfi or Tth DNA polymerases, or mutants or derivatives thereof. Taq DNA polymerase is commercially available, for example, from Life Technologies, Inc., or may be isolated from its natural source, the thermophilic bacterium Thermus aquaticus, for example, but not limited to, as described in U.S. Pat. No. 4,889,818 and U.S. Pat. No. 4,965,188. Tne DNA polymerase may be isolated from its natural source, the thermophilic bacterium Thermotoga neapolitana, for example, but not limited to as described in WO 96/10640, and Tma DNA polymerase from its natural source, the thermophilic bacterium Thermotoga maritima, for example, but not limited to as described in U.S. Pat. No. 5,374,553. Methods for producing mutants and derivatives of thermophilic DNA polymerases are also contemplated by the present invention and any such teaching known in the art may be employed to produce such mutants. Tfi, Tli/VENT™ and DEEPVENT™ are available commercially, for example, but not limited to from New England BioLabs, or may be produced as described (Bej and Mahbubani, in: PCR Technology: Current Innovations, Griffin, H. G., and Griffin, A. M., eds., CRC Press, pp. 219-237 (1994) for Tli/VENT™; Flaman, J. M., et al., Nucl. Acids Res. 22(15):3259-3260 (1994) for DEEPVENT™). Thermostable DNA polymerases are preferably added to the present compositions at a final concentration in solution of about 0.1-500 units per milliliter, about 0.1 to 100 units per milliliter, about 0.1-50 units per milliliter, about 0.1-40 units per milliliter, about 0.1-36 units per milliliter, about 0.1-34 units per milliliter, about 0.1-32 units per milliliter, about 0.1-30 units per milliliter, or about 0.1-20 units per milliliter, and most preferably at a concentration of about 20 units per milliliter. However, the present invention contemplates adding DNA polymerase at a final concentration of about 0.1, 0.5, 1, 2.5, 5, 7.5, 10, 12.5, 15, 17.5, 20, 22.5, 25, 30, 35, 40, 45, 50, 60, 70, 80, 90, 100, 125, 150, 175, 200, 250, 300, 350, 400 or 500 units per milliliter. The amount of DNA polymerase may also be defined by a range of any two of the values listed above.

In an alternate embodiment, which is not meant to be limiting in any manner, the concentration of DNA polymerases may be determined as a ratio of the concentration of the enzymes having reverse transcriptase activity.

dNTPs

The compositions of the invention may further comprise one or more nucleotides, for example, but not limited to deoxynucleoside triphosphates (dNTPs). The nucleotide components of the present compositions serve as building blocks for newly synthesized nucleic acids, being incorporated therein by the action of the reverse transcriptases or DNA polymerases. Examples of nucleotides suitable for use in the present compositions include, but are not limited to, dUTP, dATP, dTTP, dCTP, dGTP, dITP, 7-deaza-dGTP, α-thio-dATP, α-thio-dTTP, α-thio-dGTP, α-thio-dCTP or derivatives thereof, all of which are available commercially from sources including, but not limited to Life Technologies, Inc., New England BioLabs and Sigma Chemical Company (Saint Louis, Mo.). The dNTPs may be unlabeled, or they may be detectably labeled by coupling them by methods known in the art with radioisotopes such as but not limited to ³H, ¹⁴C, ³²P or ³⁵S, vitamins such as but not limited to biotin, fluorescent moieties such as but not limited to fluorescein, rhodamine, Texas Red, or phycoerythrin, chemiluminescent labels, dioxigenin and the like. Labeled dNTPs may also be obtained commercially for example, but not limited to from Life Technologies, Inc. or Sigma Chemical Company. In the present compositions, the dNTPs are added to give a working concentration of each dNTP of about 5-5000 micromolar, about 10-500 micromolar, about 10-250 micromolar, or about 10-100 micromolar, and most preferably a concentration of about 100 micromolar. However, the present invention contemplates a working concentration of dNTPs of about 5, 10, 25, 75, 100, 125, 150, 175, 200, 250, 500, 600, 700, 800, 900, 1000, 2000, 3000, 4000, 5000 micromolar. Also, the concentration used may be defined by a range of any two of the values listed above.

Primers

In addition to nucleotides, the present compositions may also comprise one or more primers which facilitate the synthesis of a first DNA molecule complementary to all or a portion of the human β-retrovirus RNA template. Such primers may also be used to synthesize a DNA molecule complementary to all or a portion of the first DNA molecule, thereby forming a double-stranded cDNA molecule. Additionally, these primers may be used in amplifying nucleic acid molecules in accordance with the invention. Such primers include, but are not limited to, human β-retrovirus specific primers, meaning that the primers are capable of hybridizing to a target human β-retrovirus RNA primer sequence and amplifying a target human β-retrovirus sequence. The primer will be completely or substantially complementary to a region of the polynucleotide strand to be copied. Thus, under conditions conducive to hybridization, the primer will anneal to the complementary region of the analyte strand, and upon addition of suitable reactants (e.g., a polymerase, nucleotide triphosphates, and the like), the primer is extended by the polymerizing agent to form a copy of the analyte strand. Additional primers that may be used for amplification of the DNA molecules according to the methods of the invention will be apparent to one of ordinary skill in the art.

Probes

The compositions of the present invention may further comprise one or more probes to monitor the PCR amplification reaction. For example, RT-PCR may employ a probe, for example, a fluorescently labelled probe to detect and quantity target sequences of DNA. In an embodiment of the present invention the probe comprises a fluorescent tag, for example a dye or the like on one end and a suitable quencher on the other. During each amplification cycle, the probe attaches to a target nucleotide sequence of human β-retrovirus RNA (or DNA made therefrom), followed by attachment of the primers to the target human β-retrovirus nucleotide sequences. As the targeted nucleotide strand is copied, the reporter dye is released from the probe and emits a fluorescent signal. She amount of fluorescence signal increases with each cycle of PCR in proportion to the amount of target DNA. This permits direct detection and quantification of the target DNA with a high degree of specificity, accuracy and sensitivity. In a preferred embodiment the RT-PCR composition, methods and kits of the present invention comprise or employ TaqMan technology to detect and quantify human β-retrovirus RNA. However, other types of probe technologies may also be employed, for example, but not limited to Molecular Beacons, Scorpions®, or SYBR® Green.

Additional Reagents

In accordance with the compositions, methods and kits of the invention, one or more additional reagents may be include or employed therein. Such reagents include, but are not limited peptides, polypeptides and proteins such as, human serum albumin, bovine serum albumin, ovalbumin, Albumax, casein, gelatin, collagen, globulin, lysozyme, transferrin, myoglobin, hemoglobin, lactalbumin, fumarase, glyceraldehyde-3-phosphate dehydrogenase (GAPDH), amyloglucosidase, carbonic anhydrase, lactoglobulin, aprotinin, soybean trypsin inhibitor, trypsinogen, phosphorylase b, myosin, actin, galactosidase, catalase, tryptic soy digests, tryptose, lectins and the like, or fragments or derivatives thereof. When added to the RT-PCR reactions, tie reagents are preferably used in low amount, for example, in the range of about 0.001 to about 100 μg/ml. In specific embodiments, not meant to be limiting in any manner, the reagents may be present in an amount of 0.005, 0.01, 0.05, 0.1, 0.5, 1, 2, 5, 10, 15, 20, 25, 50, 75 or 100 μg/ml. Alternatively, the additional reagents may be present in a range defined by any two of the values listed above.

Methods of RT-PCR

In the RT-PCR reaction, the reaction mixtures are incubated at a temperature sufficient to synthesize a DNA molecule complementary to all or portion of the human β-retrovirus RNA template. Such conditions typically range from about 20° C. to 75° C., more preferably from about 35° C. to 60° C. and most preferably from about 45° C. to about 55° C. However, the method of the present invention contemplates incubating the reaction mixture at about 20° C., 25° C., 30° C., 35° C., 40° C., 45° C., 50° C., 55° C., 60° C., 65° C. or 70° C. The present invention also contemplates that the reaction mixture may be incubated at any temperature within a range defined by any two of the values listed above.

After the reverse transcription reaction, the reaction is incubated at a temperature sufficient to amplify the synthesized DNA molecule. Preferably, the amplification is accomplished via one or more polymerase chain reactions (PCRs). Preferred conditions for amplification comprise thermocycling, which may comprise alternating heating and cooling of the mixture sufficient to amplify the DNA molecule and which most preferably comprises alternating from a first temperature range of from about 90° C. to about 100° C., to a second temperature range of from about 45° C. to about 75° C., more preferably from about 50° C. to about 75° C. or from about 55° C. to about 75° C. and most preferably from about 65° C. to about 75° C. According to the invention, the thermocycling may be performed any number of times, preferably from about 5 to about 80 times, more preferably greater than about 10 times and most preferably greater than about 20 times. However, the method of the present invention specifically contemplates thermocycling 10, 15, 20, 25, 26, 27, 28, 29, 30, 31, 32, 33, 34, 35, 36, 37, 38, 39, 40, 41, 42, 43, 44, 45, 50, 55, 75 or 100 times. Further, the amount of thermocycling may be defined by a range of any two of the values listed above.

In an embodiment of the present invention, there is provided a method for the production, detection, amplification, or quantification (or any combination thereof) of human β-retrovirus comprising the steps of:
a) obtaining a biological sample from a subject;
b) performing RT-PCR on RNA isolated from said sample and;
c) determining if said RT-PCR has amplified a nucleic acid, or fragment thereof from said human β-retrovirus.

The method as provided above may also include one or more steps relating to quantification of the human β-retrovirus nucleic acid, or fragment thereof. In such an instance, it is possible for a clinician or the like to estimate the relative amount of human β-retrovirus infection in a subject.

As provided above, the compositions and methods of the present invention may be used for diagnostic PCR analysis for the identification of retroviral nucleotides which correlate with the presence of a human β-retrovirus. In alternate embodiments, which are not meant to be limiting in any manner, the compositions and methods of the present invention may be employed for the identification of retroviral nucleotides which correlate with associated disorders, such as, but not limited to primary biliary cirrhosis (PBC), Sjogren's syndrome, scleroderma, SLE, autoimmune thyroiditis, various other connective tissue disorders and lymphoma. In yet another embodiment, the compositions and methods of the present invention can be used in therapeutic PCR analysis to monitor the presence of a human β-retrovirus and determine the effectiveness of a therapeutic protocol. For example, but not wishing to be limiting in any manner, the methods and compositions of the present invention may be employed to determine the effectiveness of antiviral or other therapy. Representative examples of antiviral drugs that may be employed in therapy include, but are not limited to lamivudine, zidovudine, Combivir or any other antiviral or combination of antiviral drugs.

It is also contemplated that subjects may be treated with ursodeoxycholic acid, alone or in combination with one or more antiviral drugs as known in the art and/or as provided above.

In an embodiment wherein lamividine is employed as an antiviral drug, generally it is used in an amount of about 50 to 300 mg per day, more preferably about 100 mg to 200 mg per day, still more preferably about 150 mg per day. In an embodiment wherein combivir combination therapy is employed as an antiviral composition, generally, it comprises about 150 mg of lamivudine and 300 mg of zidovudine per day, more preferably twice a day. In an embodiment wherein ursodeoxycholic acid (UDCA) is employed in therapy, generally it is used in an amount of about 5 to 30 mg/kg/day, more preferably about 10-20 mg/kg/day, still more preferably about 13-15 mg/kg/day.

The present invention contemplates a method of monitoring the therapy of a subject having a β-retrovirus infection comprising:
a) treating the subject with one or more therapeutics, and;
b) quantifying the level of human β-retrovirus in said subject.

The method may additionally employ the step of comparing the level of human β-retrovirus in said subject after treatment with the results of one or more prior tests wherein the level of human β-retrovirus in the subject is determined. In this regard, a particular therapy may be effective if the levels of human β-retrovirus are maintained, more preferably reduced over the course of the subject's treatment regimen. In such a manner, it is possible to monitor the effectiveness of a particular treatment.

In an alternate embodiment, there is provided a method monitoring the therapy of a subject having a β-retrovirus infection comprising:
a) identifying and/or quantifying a pretreatment level of human β-retrovirus in the subject;
b) treating the subject with one or more therapeutics, and;
c) identifying and/or quantifying the post treatment level of human β-retrovirus in the subject

In still a further embodiment, which is no, meant to be limiting, the subject may be monitored by, or subjected to one or more additional tests, for example, but not limited to liver tests, biopsies, blood tests or the like. In a preferred embodiment, the level of human β-retrovirus is monitored in combination with one or more enzyme or blood/tissue component levels, for example, but not limited to bilirubin, serum amylase, serum bicarbonate, lactate, alkaline phosphatase, aspartate aminotransferase (AST), alanine aminotransferase (ALT) or the like. It is also contemplated that the level of human β-retrovirus may be monitored in combination with one or more histological exanimations, biopsies or the like, for example, but limited to qualitative and/or quantitative examination of ductopenia, qualitative and/or quantitative examination of inflammation, for example, but not limited to necroinflammatory scores. Biopsies may be assessed for ductopenia, staged for fibrosis, graded for necroinflammatory activity, and/or evaluated for bile duct injury using the Ishak scoring system (Ishak et al., J. Hepatol 1995, 22:696-9, which is herein incorporated by reference).

In a preferred embodiment, the primers for amplifying a human β-retrovirus target sequence comprise nucleotide sequence that hybridize to the U5 long terminal repeat and pol regions of the human β-retrovirus.

The above description is not intended to limit the claimed invention in any manner, furthermore, the discussed combination of features might not be absolutely necessary for the inventive solution.

The present invention will be further illustrated in the following examples. However it is to be understood that these examples are for illustrative purposes only, and should not be used to limit the scope of the present invention in any manner.

### EXAMPLES

### Example 1

### Human β-retrovirus Sequence and Additional Information

The nucleotide sequence of human β-retrovirus has been previously described. 9690 base pairs of the proviral gyenome is provided in GenBank accession nos AF513913-AF513923. Additional information concerning the isolation and characterization of the human β-retrovirus is provided in Xu et al., PNAS July, 2003 Vol 100 No. 14 8454-8459; Mason et al., Hepatology 2004; 39:151-156; and U.S. Pat. No. 6,468,737 the disclosures of which are herein incorporated by reference.

### Example 2

### Representative Example of RT-PCR for Human β-retrovirus

Serum samples were assessed by RT-PCR for evidence of human β-retrovirus infection. RT-PCR was performed with nested oligonucleotide primers complementary to the of human β-retrovirus long terminal repeat (Xu et al., Proc. Natl. Acad Sci 2003; 100:8454-51). Total RNA was extracted from serum with Trizol (Gibco BRL, Grand Island, N.Y.), and random primers were used to make cDNA template for PCR. Nested PCR was performed using 5′-AGAAATGGTTGAACTCCCGAGAG-3′ and 5′-GGCTTGTAAGAGGAAGTTGGCTG-3′ for external primers, and 5′-TTGTTTCCCACCAAGGACGAC-3′ and 5′-GAAGGTCGAGTTCTCCGAATCG-3′ as internal primers to provide a 216 bp product. Both PCR and reactions were performed in with 2.5 mM Mg⁺² using cycling parameters of 94° C., 52° C., and 72° C. for 20s at each temperature in first PCR and for 10 s at each temperature in the nexted PCR.

### Example 3

### Representative Examples of Primers and Probes for Real Time RT-RCR of Human β-retrovirus, Nucleic Acids

Representative examples of primers and probes that may be used in real time RT-PCR are provided in Table 1. The table indicates the location of the human β-retrovirus primer or probe target sequence (i.e., U5 or Pol), forward or reverse primers, the 5′ to 3′ sequence of the primers/probe, amplicon in base pairs, location thereof and gene including GenBank accession number. A person of skill in the art will recognize that a variety of other primers and probes may be used in real time RT-PCR.

TABLE 1

Sequences of the primers and probes for detection
and quantification of human betaretrovirus

| Name of primes | Sequence | Amplicon | | |
|---|---|---|---|---|
| and probes | (5′-3′) | (bp) | Location | Gene (clone) |
| U5-F | GCCCATCAGACAAAGACATACTCA | 72 | 44-67 | U5(AF513913) |
| U5-R | CCAATAGCCCCAGGCAAA | | 115-98 | U5(AF513913) |
| U5-probe | TCTCTGCTGCAAACTTGGCATAGCTCTG | | 69-96 | U5(AF513913) |
| Pol-F1 | TCACATGACCTCTACCACACCACTA | 74 | 398-422 | Pol(AF513922) |
| Pol-R1 | CCGAACGGCCATTTGC | | 471-456 | Pol(AF513922) |
| Pol-probe1 | AGAAAGGAATTGTGATTTTTACGGACGGGTC | | 424-454 | Pol(AF513922) |
| Pol-F2 | GCTGTTACAAGACCTACGTGCAGTT | 71 | 1161-1185 | Pol(AF248269) |
| Pol-R2 | ACGGCAAGCCTGGTTGTAAT | | 1231-1212 | Pol(AF248269) |
| Pol-probe2 | TGCCACAATGCACGATATGGGA | | 1188-1209 | Pol(AF248269) |

### Example 4

### Human β-retrovirus Specific Real Time RT-PCR Assay

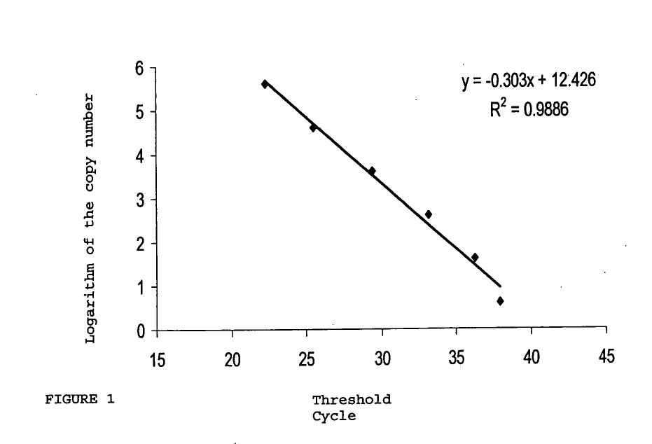

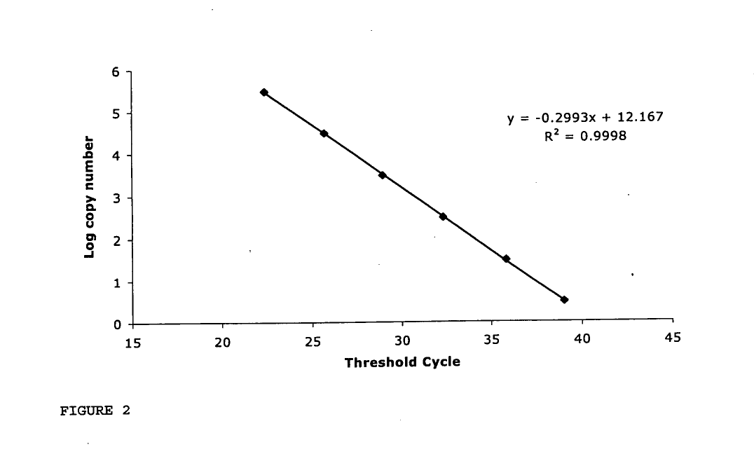

Two quantitative Real Time RT-PCR assay s have been developed using the TaqMan technology to detect and quantify human β-retrovirus RNA. Primers and probe complementary to the U5 long terminal repeat and pol regions of the human β-retrovirus as discussed above were employed. To assess the sensitivity of each assay, 10-fold serial dilutions of a plasmid containing the human β-retrovirus long terminal repeat and pol regions were prepared using an approximate range of 4×10⁵ to 4 DNA copy number (FIGS. 1 and 2). Each assay has the capability to detect less than about 10 plasmid copies.

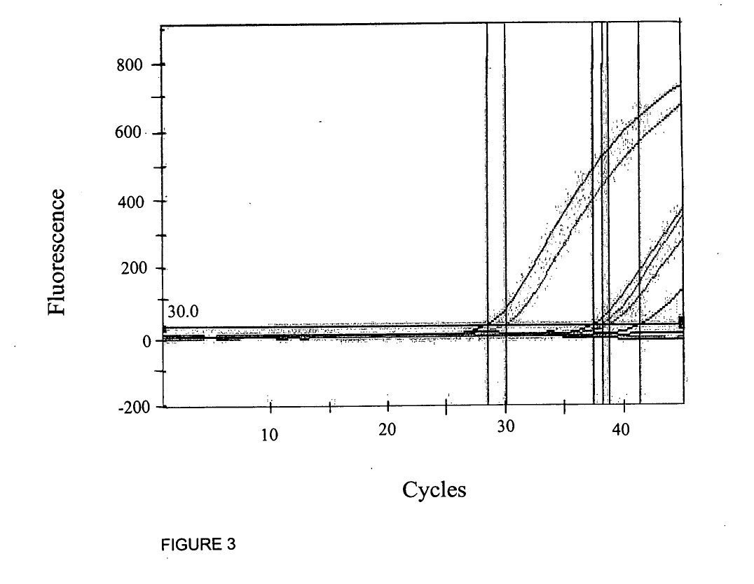

Preliminary studies revealed that both assays are functional and complementary with regard to detecting viral levels in tissue and serum of patients with PBC. It has been observed that human β-retrovirus RNA in the liver is detected in a 10³ higher viral load compared to serum from subjects with PBC. Also the appropriate control subjects either have low or undetectable viral load (FIG. 3).

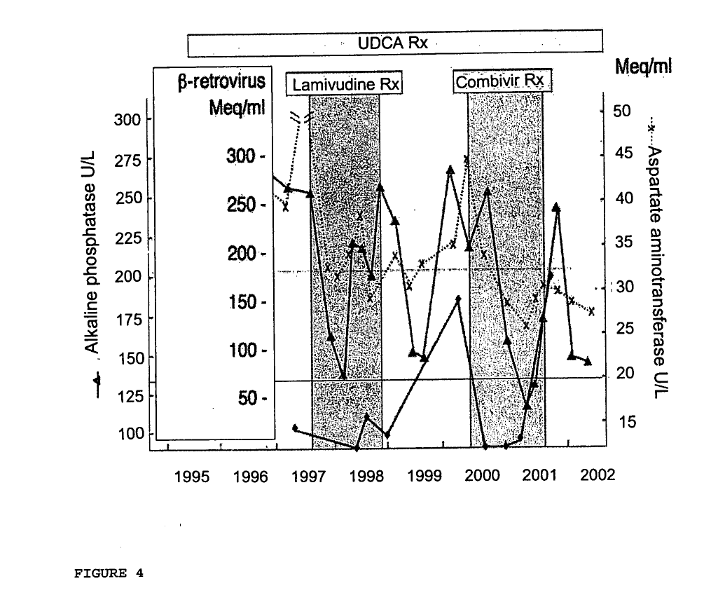

Preliminary studies have been conducted to determine whether the real-time RT-PCR assays will be functional to measure viral load in response to antiviral treatment. To date, we have observed that most patients have biochemical responses with improvement in the liver function tests, alkaline phosphatase and alanine aminotransferase (ALT) corresponding with the initiation of antiviral therapy. By testing cDNA from serial serum samples derived from patients undergoing lamivudine, or Combivir therapy, we have been able to verify the utility of both assays by demonstrating that the reduction of viral levels corresponding with antiviral treatment and improvement in liver biochemistry studies (FIG. 4).

All citations are herein incorporated by reference.

The present invention has been described with regard to preferred embodiments. However, it will be obvious to persons skilled in the art that a number of variations and modifications can be made without departing from the scope of the invention as described herein.

What is claimed is:

1. A method of monitoring the therapy of a subject having a β-retrovirus infection comprising,
a) treating the subject with one or more therapeutics, and;
b) quantifying the level of human β-retrovirus in said subject.

2. The method of claim 1, wherein said quantifying is performed by real time RT-PCR.

3. The method of claim 2, wherein said one or more therapeutics comprise lamivudine, zidovudine, combivir, ursodeoxycholic acid or a combination thereof.

4. The method of claim 2, wherein said real time RT-PCR employs a primer and probe as defined in Table 1.

5. The method of claim 2, wherein said RT-PCR is performed on nucleic acids obtained from a biological sample.

6. The method of claim 5, wherein said biological sample is a tissue, fluid or combination thereof isolated from a human subject.

7. The method of claim 6, wherein said biological sample is blood plasma, serum, spinal fluid, lymph fluid, or a combination thereof.

8. The method of claim 7, wherein said biological sample is liver tissue from a biopsy.

9. A method of monitoring the therapy of a subject having or suspected of having a human β-retrovirus infection comprising:
a) quantifying one or more pretreatment levels of human β-retrovirus in said subject;
b) treating said subject with one or more therapeutics, and;
c) quantifying one or more post-treatment level of human β-retrovirus in said subject.

10. The method of claim 9, wherein during said b) treating said subject with one or more therapeutics, one or more levels of one or more levels of human β-retrovirus is determined.

11. The method of claim 1, wherein the level of human β-retrovirus is monitored in combination with one or more enzyme or blood/tissue component levels.

12. The method of claim 11, wherein the one or more enzyme or blood/tissue component levels include bilirubin, serum amylase, serum bicarbonate, lactate, alkaline phosphatase, aspartate aminotransferase (AST), alanine aminotransferase (ALT) or a combination thereof.

13. A method of screening a compound or composition for anti-human β-retrovirus activity comprising,
a) administering the compound or composition to one or more subjects having human β-retrovirus;
b) quantifying the level of human β-retrovirus in said subject.

14. The method of claim 13, wherein human β-retrovirus infection is detected and/or quantified prior to said a) administering the compound or composition to one or more subjects.

15. A kit for detecting and/or quantifying human β-retrovirus in a biological sample comprising,
a) one or more primers for amplifying a human β-retrovirus target sequence,
b) one or more reverse transcriptases,
c) one or more DNA polymerases,
d) one or more probes for performing real time RT-PCR,
e) one or more buffers for performing, terminating, inhibiting, diluting, washing one or more enzymatic reactions, or products produced purified in a RT-PCR reaction;
f) one or more restriction enzymes for cleaving DNA amplified from a PCR reaction,
g) one or more hybridization probes for identifying DNA amplified from a PCR reaction,
h) instructions for performing a RT-PCR reaction using the components of the kit,
i) one or more therapeutics;
j) one or more dNTPs, or the like,
or any combination thereof.

16. The kit of claim 15, wherein said one or more primers comprise nucleotide sequence that hybridize to the U5 long terminal repeat or pol regions of the human β-retrovirus.

17. The kit of claim 16, wherein said primers are as defined in Table 1.


SEQUENCE LISTING (13 sequences)

SEQ ID NO: 1 - DNA, 23 bp, Artificial Sequence
  External primer
agaaatggttgaactcccgagag

SEQ ID NO: 2 - DNA, 23 bp, Artificial Sequence
  External primer
ggcttgtaagaggaagttggctg

SEQ ID NO: 3 - DNA, 21 bp, Artificial Sequence
  Internal primer
ttgtttcccaccaaggacgac

SEQ ID NO: 4 - DNA, 22 bp, Artificial Sequence
  Internal primer
gaaggtcgagttctccgaatcg

SEQ ID NO: 5 - DNA, 24 bp, Artificial Sequence
  U5-F
gcccatcagacaaagacatactca

SEQ ID NO: 6 - DNA, 18 bp, Artificial Sequence
  U5-R
ccaatagccccaggcaaa

SEQ ID NO: 7 - DNA, 28 bp, Artificial Sequence
  U5-probe
tctctgctgcaaacttggcatagctctg

SEQ ID NO: 8 - DNA, 25 bp, Artificial Sequence
  Pol-F1
tcacatgacctctaccacaccacta

SEQ ID NO: 9 - DNA, 16 bp, Artificial Sequence
  Pol-R1
ccgaacggccatttgc

SEQ ID NO: 10 - DNA, 31 bp, Artificial Sequence
  Pol-probe1
agaaaggaattgtgatttttacggacgggtc

SEQ ID NO: 11 - DNA, 25 bp, Artificial Sequence
  Pol-F2
gctgttacaagacctacgtgcagtt

SEQ ID NO: 12 - DNA, 20 bp, Artificial Sequence
  Pol-R2
acggcaagcctggttgtaat

SEQ ID NO: 13 - DNA, 22 bp, Artificial Sequence
  pol-probe2
tgccacaatgcacgatatggga